(19) United States
(12) Patent Application Publication
(10) Pub. No.: US 2008/0033065 A1
(43) Pub. Date: Feb. 7, 2008

(54) SYSTEM AND METHOD FOR ENHANCING CO PRODUCTION IN A RICH COMBUSTION SYSTEM

(75) Inventor: JOEL MEIER HAYNES, NISKAYUNA, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: GENERAL ELECTRIC COMPANY, SCHENECTADY, NY (US)

(21) Appl. No.: 11/462,475

(22) Filed: Aug. 4, 2006

(51) Int. Cl. C07C 27/06

(52) U.S. Cl. 518/702

(57) ABSTRACT

A device for enhancing carbon monoxide (CO) production in an exhaust gas generated from a system is provided. The device includes a flow path configured to direct the exhaust gas from the system into a heat treatment zone and an energy source configured to provide local heat treatment to the exhaust gas in the heat treatment zone for shifting an equilibrium point of reaction substantially away from carbon dioxide (CO2) formation thereby promoting formation of an increased level of carbon monoxide (CO) in the exhaust gas.

### BACKGROUND

The invention relates generally to a system for reforming of an exhaust gas, and more particularly to a system for enhancing carbon monoxide (CO) formation in synthetic gas.

Currently industrial plants are built around the globe to produce synthesis gas for use in a variety of applications including conversion of natural gas to useful liquid fuels, generation of hydrogen-enriched gases, production of dimethylether (DME), methanol, and other processes. Typically, synthesis gases produced in a gas to liquid plant are supplied to a Fischer Tropsch processing unit for catalytically converting the quenched synthesis gas into a long-chain hydrocarbon fluid. Further, the long-chain hydrocarbon fluid mixture is fractionated into at least one useful product through an upgrading process.

In certain traditional systems, synthesis gases are produced through diffusion combustion of reactants or through an auto thermal reformer (ATR), or through a premixed reaction zone. Unfortunately, the diffusion combustion requires a substantially long residence time to ensure that the products of the diffusion flame achieve near equilibrium products at the exit of a syngas generator. Furthermore, an ATR requires large amounts of steam and has limited life. In addition, partial premixing of the reactants may reduce the residence time but may not provide the desired conversion efficiency.

Accordingly, there is a need for a system that has a high conversion efficiency of natural gas to syngas products. Furthermore, it would be desirable to provide a system that will utilize natural gas and oxygen effectively to produce syngas having an enhanced CO production.

### BRIEF DESCRIPTION

Briefly, according to one embodiment, a device for enhancing carbon monoxide (CO) production in an exhaust gas generated from a system is provided. The device includes a flow path configured to direct the exhaust gas from the system into a heat treatment zone and an energy source configured to provide local heat treatment to the exhaust gas in the heat treatment zone for shifting an equilibrium point of reaction substantially away from carbon dioxide (CO2) formation thereby promoting formation of an increased level of carbon monoxide (CO) in the exhaust gas.

In another embodiment, a method of producing synthesis gas is provided. The method includes premixing a fuel stream and oxidizer to form a gaseous premix in a premixing zone and locally heating the gaseous premix downstream of the premixing zone. The method also includes combusting the gaseous premix in a combustion zone to form syngas enriched with carbon monoxide (CO).

In another embodiment, a system for producing syngas is provided. The system includes a premixing device configured to mix a fuel stream and an oxidizer in a premixing zone to form a gaseous pre-mix and an energy source coupled to the premixing device and configured to locally shift the equilibrium point of the gaseous pre-mix in a heat treatment zone disposed downstream of the premixing zone. The system also includes a combustion zone configured to receive the gaseous pre-mix from the heat treatment zone and to combust the gaseous premix to produce syngas enriched with carbon monoxide (CO).

### DRAWINGS

These and other features, aspects, and advantages of the present invention will become better understood when the following detailed description is read with reference to the accompanying drawings in which like characters represent like parts throughout the drawings, wherein:

FIG. 1 is a diagrammatical illustration of a gas to liquid system having a syngas generator in accordance with aspects of the present technique.

FIG. 2 is a diagrammatical illustration of an exemplary configuration of a syngas generator employed in the gas to liquid system of FIG. 1 in accordance with aspects of the present technique.

FIG. 3 is a diagrammatical illustration of another exemplary configuration of a syngas generator with staged heat treatment employed in the gas to liquid system of FIG. 1 in accordance with aspects of the present technique.

FIG. 4 is a diagrammatical illustration of exemplary configurations of the syngas generator of FIG. 2 having a plasma arc as an energy source in accordance with aspects of the present technique.

FIG. 5 is a diagrammatical illustration of exemplary configurations of the syngas generator of FIG. 2 having a high energy laser as an energy source in accordance with aspects of the present technique.

FIG. 6 is a diagrammatical illustration of an exemplary trapped vortex configuration employed in the syngas generator of FIG. 2 in accordance with aspects of the present technique.

FIG. 7 illustrates exemplary configurations for achieving a staged heat treatment of an exhaust gas such as syngas generated from the syngas generator of FIG. 3 in accordance with aspects of the present technique.

FIG. 8 illustrates exemplary configurations for achieving local reforming of a gas stream in the syngas generator of FIG. 1 in accordance with aspects of the present technique.

FIG. 9 illustrates an exemplary configuration for achieving a closed-loop control of fuel reforming based upon a sensed parameter in accordance with aspects of the present technique.

FIG. 10 illustrates an exemplary configuration to detect an operational condition in a system such as the syngas generator of FIG. 1 in accordance with aspects of the present technique.

FIG. 11 illustrates another exemplary configuration to detect an operational condition in a system such as the syngas generator of FIG. 1 in accordance with aspects of the present technique.

FIG. 12 illustrates an internal combustion engine system having an energy source for achieving fuel reforming of exhaust products from the engine in accordance with aspects of the present technique.

FIG. 13 illustrates an exemplary configuration for flame detection in a system such as the syngas generator of FIG. 1 in accordance with aspects of the present technique.

### DETAILED DESCRIPTION

As discussed in detail below, embodiments of the present technique function to achieve reforming of an exhaust gas by enhancing carbon monoxide (CO) in the exhaust gas generated from a rich combustion system. In particular, the present technique employs a local heat treatment in such systems for shifting an equilibrium point of the reaction substantially away from carbon dioxide (CO₂) thereby promoting formation of carbon monoxide in the exhaust gas. As discussed in detail below, the technique may be employed to enhance the conversion efficiency of syngas generators for producing a syngas composition enriched with carbon monoxide. Furthermore, the local heat treatment of the exhaust gas may also be employed to facilitate dynamics stabilization, diagnostics and control of such systems.

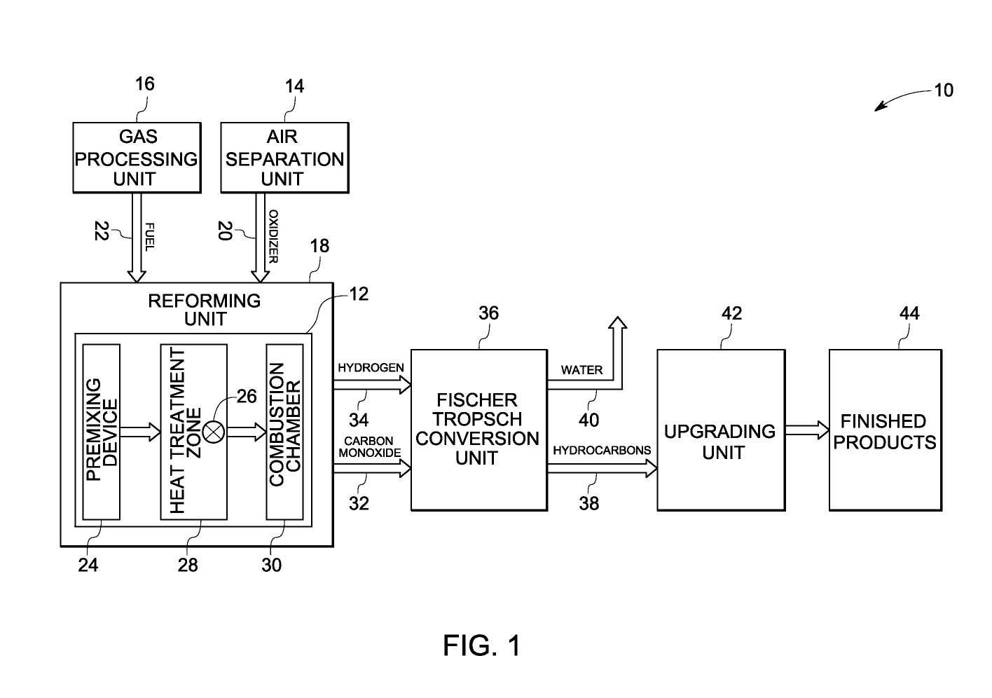

Turning now to the drawings and referring first to FIG. 1 a gas to liquid system 10 having a syngas generator 12 is illustrated. The gas to liquid system 10 typically includes an air separation unit 14 and a gas processing unit 16. The air separation unit 14 separates air into nitrogen (N₂), oxygen (O₂) and other gases. Further, the gas processing unit 16 is configured to prepare a fuel stream for combustion. In particular, the gas processing unit 16 prepares raw natural gas for conversion in a reforming unit 18 by filtering and reducing the levels of impurities such as sulfur.

In the illustrated embodiment, the reforming unit 18 includes the syngas generator 12 for reacting an oxidizer such as oxygen 20 and a fuel stream 22 from the air separation and gas processing units 14 and 16, respectively, to produce a synthesis gas. In the illustrated embodiment, the syngas generator 12 includes a premixing device 24 that is configured to mix the fuel stream 22 and oxygen 20 to form a gaseous pre-mix. In certain embodiments, the fuel stream 22 and oxygen 20 are preheated prior to mixing in the premixing device 24. Further, the syngas generator 12 includes an energy source 26 to increase a temperature of the gaseous pre-mix in a heat treatment zone 28 disposed downstream of the premixing device 24. Advantageously, the localized heat treatment of the gaseous pre-mix prior to combustion enhances the conversion efficiency of the syngas generator 12. Examples of the energy source 26 include one ore more plasma arcs, a high energy laser, a leaner pilot and combinations thereof. In certain embodiments, the temperature of the gaseous pre-mix may be increased via a trapped vortex configuration that will be described in detail below.

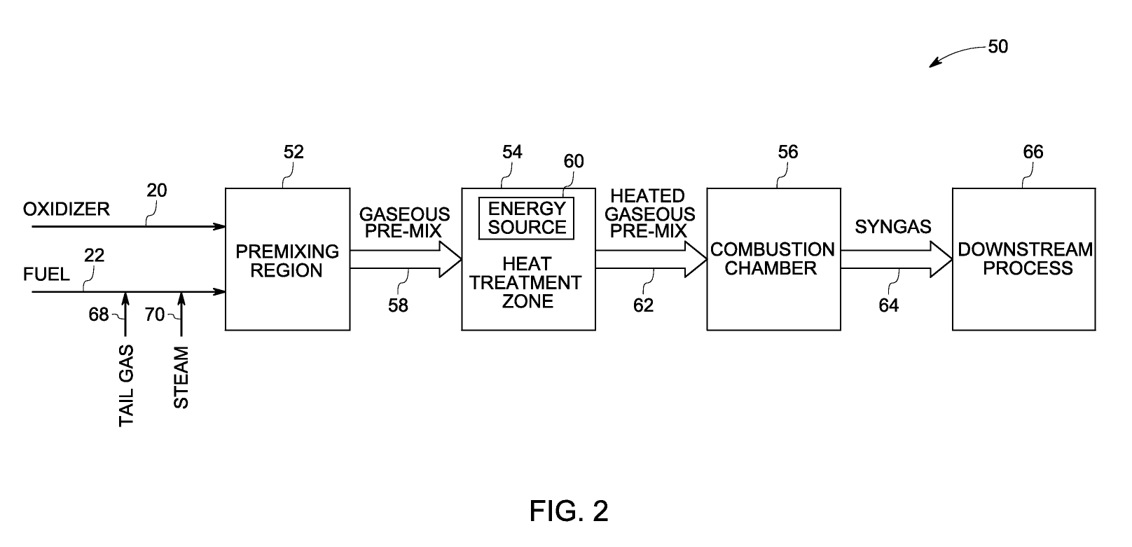

Further, the syngas generator 12 includes a combustion chamber 30 configured to combust the gaseous pre-mix from the heat treatment zone 28 to produce synthesis gas enriched with carbon monoxide 32. In certain embodiments, the combustion chamber 32 may have a substantially shorter residence time than that of a traditional partial oxidation (POX) reactor. Furthermore, in certain embodiments, a turbo expander may be disposed at an exit of the reforming unit 18 to power the air separation unit 14. The gas to liquid system 10 includes a Fischer-Tropsch processing unit 36 for receiving quenched synthesis gas from the reforming unit 18 and for catalytically converting the quenched synthesis gas into hydrocarbons 38 and water 40. In addition, the gas to liquid system 10 includes an upgrading unit 42 for fractionating the hydrocarbons 38 from the Fischer Tropsch conversion unit 36 into at least one useful product 44. Examples of product 44 include synthetic diesel fuel, synthetic kerosene, ethanol, dimethyl ether, naptha and combinations thereof. In the illustrated embodiment, the heat treatment zone 28 is disposed at an exit of the premixing device 24 for heating the premixed reactants. In certain embodiments, the heat treatment zone 28 may be disposed at an exit of the combustion chamber 30 for heating the formed syngas. In accordance with the present techniques, the gas to liquid system 10 employs premixed partial oxidation combustion coupled with a localized heat treatment that will be described below with reference to FIGS. 2-6.

FIG. 2 is a diagrammatical illustration of an exemplary configuration of a syngas generator 50 employed in the gas to liquid system 10 of FIG. 1. In this exemplary embodiment, the syngas generator 50 includes a premixing region 52, a heat treatment zone 54 and a combustion chamber 56. In operation, preheated oxidizer 20 and preheated fuel stream 22 are mixed in the premixing region 52 via a premixing device 24 (see FIG. 1) to form a gaseous pre-mix 58. In one embodiment, the oxidizer 20 comprises oxygen and the fuel stream 22 comprises natural gas. Further, the gaseous pre-mix 58 formed in the premixing region 52 is directed to the heat treatment zone 54. In this embodiment, the heat treatment zone 54 includes an energy source 60 for heating the gaseous pre-mix 58 received from the premixing region 52 to form heated gaseous pre-mix 62. In this exemplary embodiment, combustion of the pre-mix 62 may be initiated before the end of the heat treatment zone 54 and continues into the combustion chamber 56 where the products are combusted at elevated temperature and pressure to form synthesis gas 64, which in turn, is directed to a downstream process 66 for further processing.

In certain embodiments, a tail gas 68 may be added to the fuel stream 22 to improve the overall conversion efficiency of the gas to liquid system 10. The tail gas 68 may include a fuel-bearing gas that is recycled from the downstream process 66. For example, in one embodiment in the gas to liquid system 10 (see FIG. 1) the tail gas is a gas phase product from the Fischer Tropsch processing unit 36 (see FIG. 1). Similarly, in certain other embodiments, the fuel stream 22 may be augmented with steam 70 to control the H2:CO ratio of the generated syngas. Further, the steam 70 may also be used to regulate the syngas temperature. In this exemplary embodiment, the localized heat addition in the heat treatment zone 54 is achieved via the energy source 60 such as a plasma arc, or a high energy laser, or any other energy source configured to achieve the local temperature rise of the gaseous pre-mix 58. In certain other embodiments, such temperature rise of the gaseous pre-mix 58 may be achieved through a leaner pilot flame in a trapped vortex configuration.

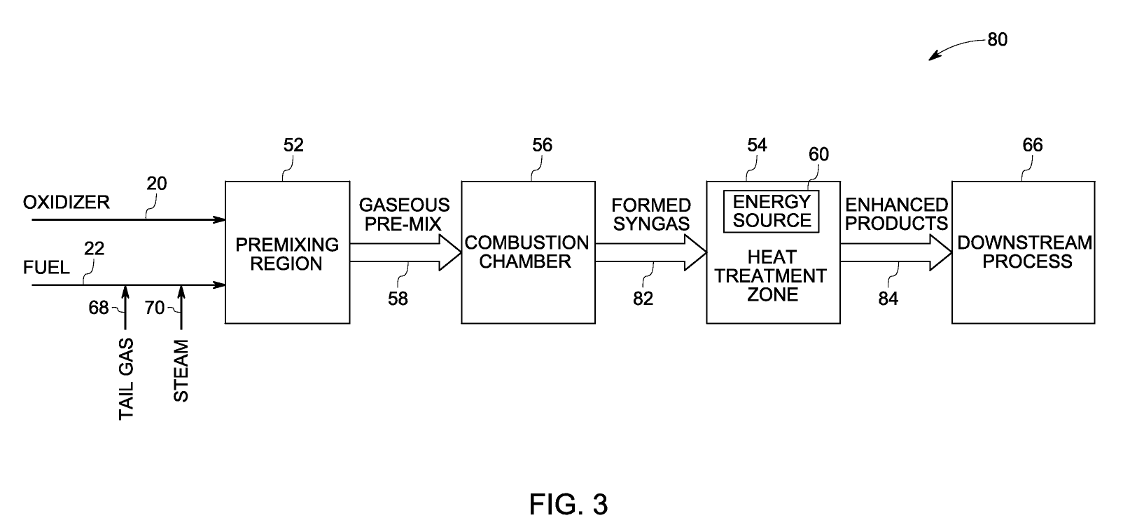

As illustrated, the heat treatment zone 54 is disposed downstream of the premixing region 52 for providing localized heating before combustion of the gaseous pre-mix 58 in the combustion chamber 56. Alternatively, the heat treatment zone 54 may be disposed downstream of the combustion chamber 56 for controlling the carbon monoxide concentration in the formed syngas 64 as described below with reference to FIG. 3.

FIG. 3 is a diagrammatical illustration of another exemplary configuration 80 of a syngas generator with staged heat treatment employed in the gas to liquid system of FIG. 1. In this exemplary configuration 80, the heat treatment zone 54 is disposed downstream of the combustion chamber 56 and is configured to provide localized heat addition to formed syngas 82 from the combustion chamber 56 to form enhanced products 84 that have relatively higher carbon monoxide concentration. The enhanced products 84 may be subsequently directed to the downstream process 66 for further processing. It should be noted that, syngas formed by other systems such as a gasifier may be subjected to such local reforming for producing enhanced products.

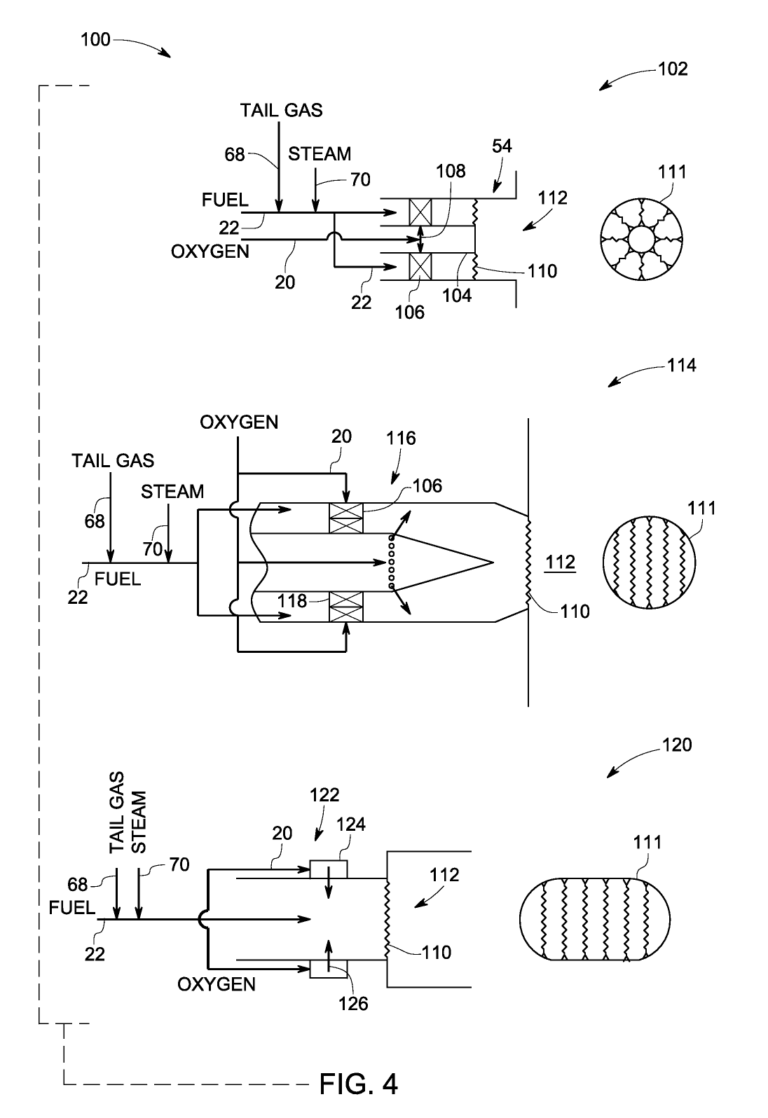

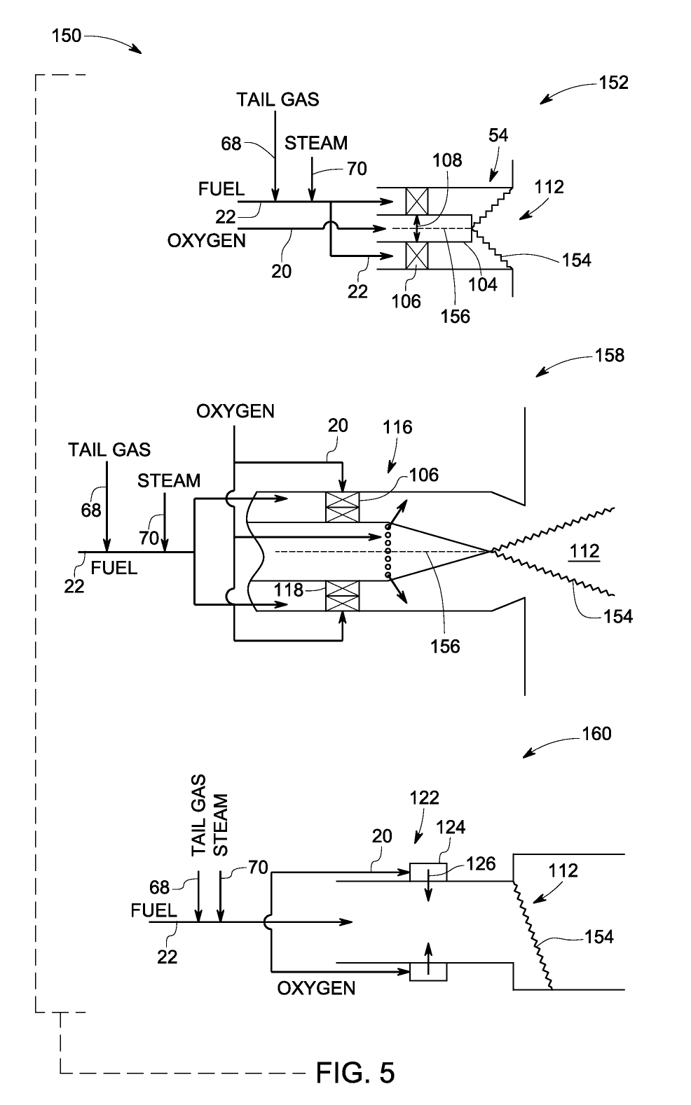

As described above with reference to FIG. 2, the heat treatment zone 54 employs an energy source 60 for heating the gaseous pre-mix 58 prior to combustion in the combustion chamber 56. Examples of energy source include plasma arc and a high energy laser. FIGS. 4 and 5 illustrate exemplary configurations of the syngas generator 50 of FIG. 2 with plasma arc and laser for achieving the localized heat treatment of the premixed reactants prior to combustion.

FIG. 4 is a diagrammatical illustration of exemplary configurations 100 of the syngas generator 50 of FIG. 2 having a plasma arc as the energy source 60. For example, in an exemplary configuration 102, the fuel stream 22 is introduced in a premixing device 104. Further, the fuel stream 22 is pre-conditioned via a flow conditioning device 106. In this exemplary embodiment, the flow conditioning device 106 includes swirler vanes. Further, oxygen 20 is introduced within the premixing device 104 through the swirler vanes 106 in a transverse direction to the direction of fuel stream 22, as represented by reference numeral 108. Again, as described before, the fuel stream 22 may be augmented by tail gas 68 or steam 70. The fuel stream 22 and oxygen 20 are premixed to form the gaseous pre-mix 58 (see FIG. 2) that is further directed to the heat treatment zone 54. In operation, the heat treatment zone 54 employs one or more plasma arcs 110 in the vicinity of the exit of the premixing device 104 for heating the gaseous pre-mix 58 to increase a temperature of the gaseous pre-mix 58. The electrical arc 110 creates an ionized gas and plasma 111 facilitates the localized shift of equilibrium for the gaseous pre-mix 58 in the heat treatment zone 54. Advantageously, the plasma 111 shifts the equilibrium point away from carbon dioxide formation thereby promoting formation of more carbon monoxide. Subsequently the pre-mix 58 is directed to the combustion chamber 56 (see FIG. 2) through an exit 112 to obtain the desired amount of hydrocarbon conversion.

In another exemplary configuration 114 of the syngas generator 50 of FIG. 2, the fuel stream 22 is introduced in a premixing device 116 and is pre-conditioned via the swirler vanes 106. Further, the premixing device 116 also includes counter flow swirl vanes 118 disposed adjacent to the plurality of swirler vanes 106. As illustrated, oxygen 20 is introduced through the swirler vanes 106 and is mixed with the pre-conditioned fuel stream 22 to form the gaseous pre-mix 58. Further, the gaseous pre-mix 58 is provided with a localized heat treatment at the exit of the premixing device 116 via one or more plasma arcs 110 and is subsequently directed to the combustion chamber 56 to react more completely for forming syngas. It should be noted that the mixing region could be either straight or converging prior to the exit 112. Further, oxygen 20 can also be introduced through the centerbody of the premixing device 116 with an aerodynamic tip to prevent flow separation or through holes in the burner tube.

Further, in an exemplary configuration 120, the fuel stream 22 is similarly introduced within a premixing device 122. Further, oxygen 20 is injected through holes 124 disposed on the burner tube, as represented by reference numeral 126. In particular, the oxygen 20 is injected through the burner tube in a transverse direction to the direction of the fuel stream 22. Again, the plasma arc 110 is employed at an exit of the device 122 for achieving the local temperature rise prior to combustion of the gaseous pre-mix 58 in the combustion chamber 56.

FIG. 5 is a diagrammatical illustration of exemplary configurations 150 of the syngas generator 50 of FIG. 2 having a high energy laser as the energy source 60. In an exemplary configuration 152, the gaseous pre-mix 58 is formed in a similar manner as described above with reference to configuration 102 of FIG. 4. However, in this exemplary embodiment, the heat treatment zone 54 employs a high energy laser for heating the gaseous pre-mix 58. In operation, a laser light sheet 154 is transmitted from a laser source (not shown) to an exit of the premixing device 104 through a fiber optic cable 156 to facilitate local heat addition at the exit for increasing the carbon monoxide formation.

Similarly, in exemplary configurations 158 and 160, the gaseous pre-mix 58 is formed by premixing fuel 22 and oxygen 20 as described above with reference to configurations 114 and 120 of FIG. 4. Again, the gaseous pre-mix 58 is provided with a localized heat treatment at the exit of the premixing device 116 through the high energy source that generates the laser light sheet 154. Subsequently, the gaseous pre-mix 58 is combusted in the combustion chamber 56 to form syngas.

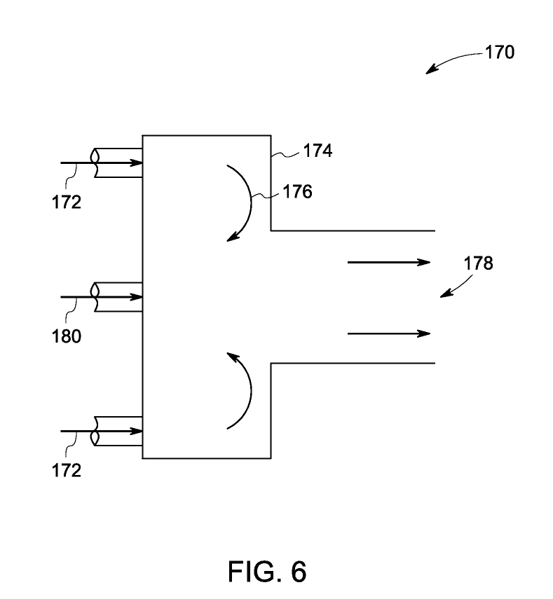

The localized heat treatment of the gaseous pre-mix 58 in the heat treatment zone 54 disposed downstream of the premixing region 52 (see FIG. 2) may be achieved through a variety of configurations with an energy source such as described above. In certain embodiments, the localized heat addition may be achieved by a leaner pilot in a trapped vortex configuration as will be described below with reference to FIG. 6.

FIG. 6 is a diagrammatical illustration of an exemplary trapped vortex configuration 170 employed in the syngas generator 50 of FIG. 2 for providing a localized heat treatment to the gaseous pre-mix 58. In this exemplary embodiment, premixed fuel and oxygen reactants 172 are introduced from the premixing zone 52 (see FIG. 2) or may be introduced independently to a trapped vortex cavity 174. In operation, the trapped vortex cavity 174 is utilized to produce an annular rotating vortex 176 of the fuel and oxygen mixture. The trapped vortex cavity 174 is designed such that it establishes the vortex 176 that feeds central burner 178 with hot products. In the illustrated embodiment, rich premixed reactants 180 are introduced in the central burner 178 through radial slots (not shown) distributed around the center of an inlet face. In certain embodiments, the central burner 178 may also be fed with these reactants in a partially premixed or unmixed manner. Further, the premixed reactants 172 are introduced within the vortex cavity 174 through ports (not shown) distributed around the outer circumference of the inlet face. Advantageously, the trapped vortex configuration 170 enables local heat addition at an exit of the premixing zone 52 by mixing the hot products from the vortex cavity 174 with the rich premixed reactants 180. Advantageously, this leads to a rise in temperature of the reactants and an increase in CO formation. Furthermore, the trapped vortex configuration 170 also enables the system to operate at richer conditions than a standard premixer due to the stabilizing characteristics of the cavity 174. It should be noted that the central burner 178 is run very rich while the cavity 174 is operated at a relatively leaner condition.

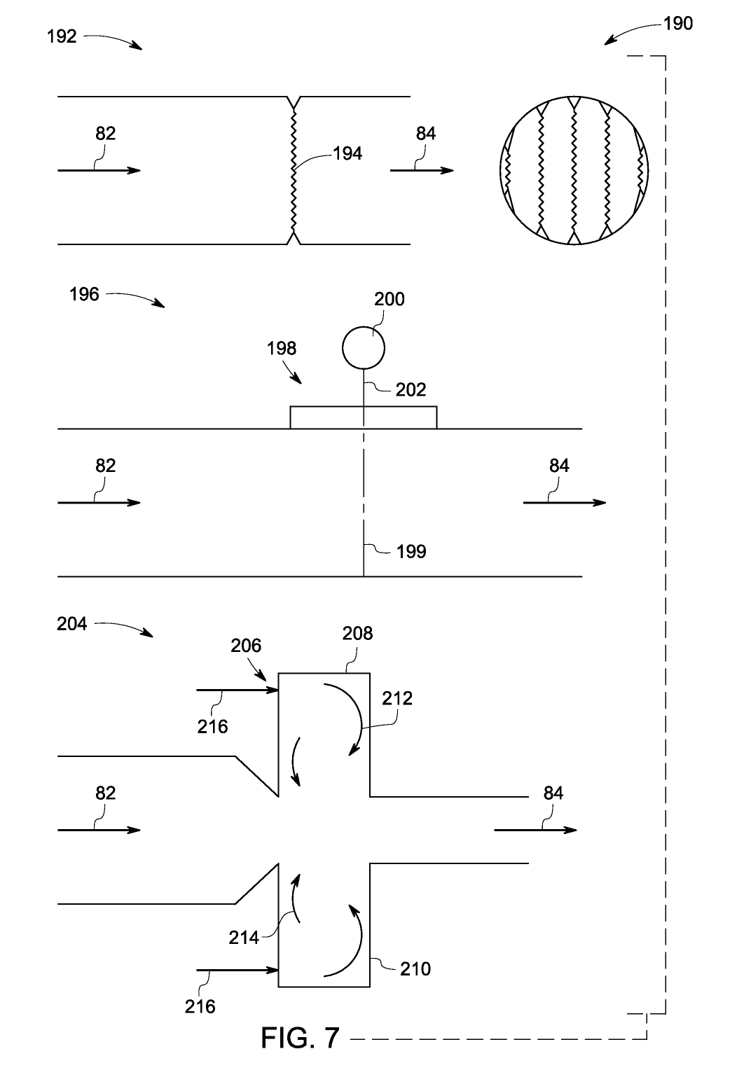

As discussed above, the gaseous pre-mix 58 is heated in the heat treatment zone 54 through an energy source 60 at an exit of the premixing region 52 to increase carbon monoxide. In certain embodiments, a staged heat treatment of formed exhaust gas such as syngas may be employed to produce enhanced products. FIG. 7 illustrate exemplary configurations 190 for achieving a staged heat treatment of an exhaust gas such as syngas 82 generated from the syngas generator 80 of FIG. 3. In one exemplary configuration 192, formed syngas 82 from the syngas generator 80 is subjected to a localized heat addition via plasma arc 194 in the heat treatment zone 54 disposed downstream of the syngas generator 12. Advantageously, the local temperature rise shifts the equilibrium point away from carbon dioxide thereby promoting formation of more carbon monoxide in the enhanced products 84. Such products 84 may be then directed to the downstream process 66 (see FIG. 3) for further processing.

Further, in the embodiment illustrated in configuration 196, the heating of the formed syngas 82 is achieved through a high-energy laser 198. In particular, a laser sheet 199 generated from a source 200 is directed to the heat treatment zone 54 through a fiber optic cable 202 for heating the syngas 82 for generating the enhanced products 84 with a relatively higher carbon monoxide concentration. In an alternate embodiment illustrated by configuration 204, a trapped vortex configuration 206 is employed to achieve the localized heating of the syngas 82. As illustrated, the exemplary configuration 206 includes two vortex cavities 208 and 210. Each of these cavities is configured to produce an annular rotating vortex such as represented by reference numerals 212 and 214 of fuel and oxygen mixture 216. As described earlier, with reference to FIG. 6, the trapped vortex configuration 206 enables local heat addition thereby promoting formation of more carbon monoxide in the enhanced products 84.

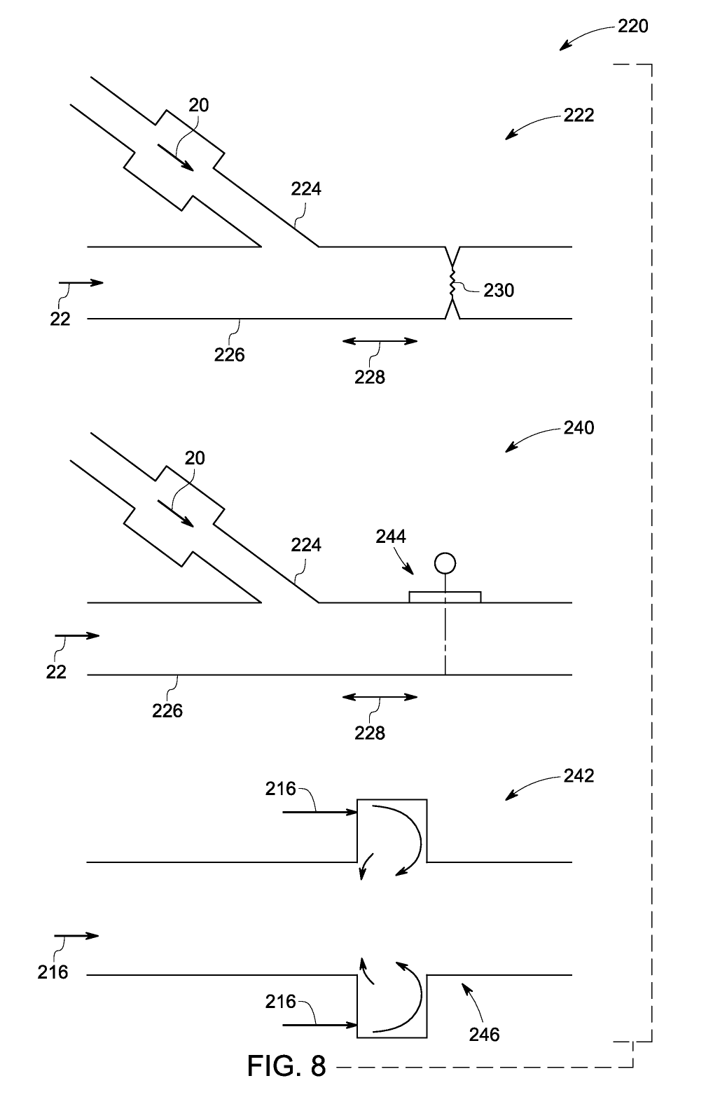

The exemplary configurations 192, 196 and 204 described above facilitate staged heat treatment of syngas via an energy source such as plasma arc, laser or through a trapped vortex configuration. In certain embodiments, the technique described above may be employed for local reforming of a gas stream such as described below with reference to FIG. 8.

FIG. 8 illustrates exemplary configurations 220 for achieving local reforming of a gas stream in a system such as syngas generator of FIG. 1. In an exemplary configuration 222, the fuel stream 22 is introduced and mixed with the oxidant 20 that is introduced through an inlet 224 located downstream of a fuel inlet 226. Further, the fuel stream 22 and the oxidant 20 are mixed in a mixing region 228 to form a pre-mix that is further subjected to a localized heat treatment through a plasma arc 230. It should be noted that the plasma arcs 230 should be separated adequately from the inlet 224 to facilitate mixing of the fuel stream 22 and the oxidant 20 prior to the heat treatment. In this exemplary embodiment, combustion occurs downstream of the plasma arcs 230 and the passage leads to the combustion chamber 30. In exemplary configurations 240 and 242, the localized heat addition is achieved through a laser 244 and a trapped vortex configuration 246 that function to provide the heat as described above with reference to FIG. 7. Again, in these configurations, the energy source such as the laser 244, or the trapped vortex configuration 246 may be spaced at an adequate distance from the inlet 224 to provide adequate length of premixing region 228.

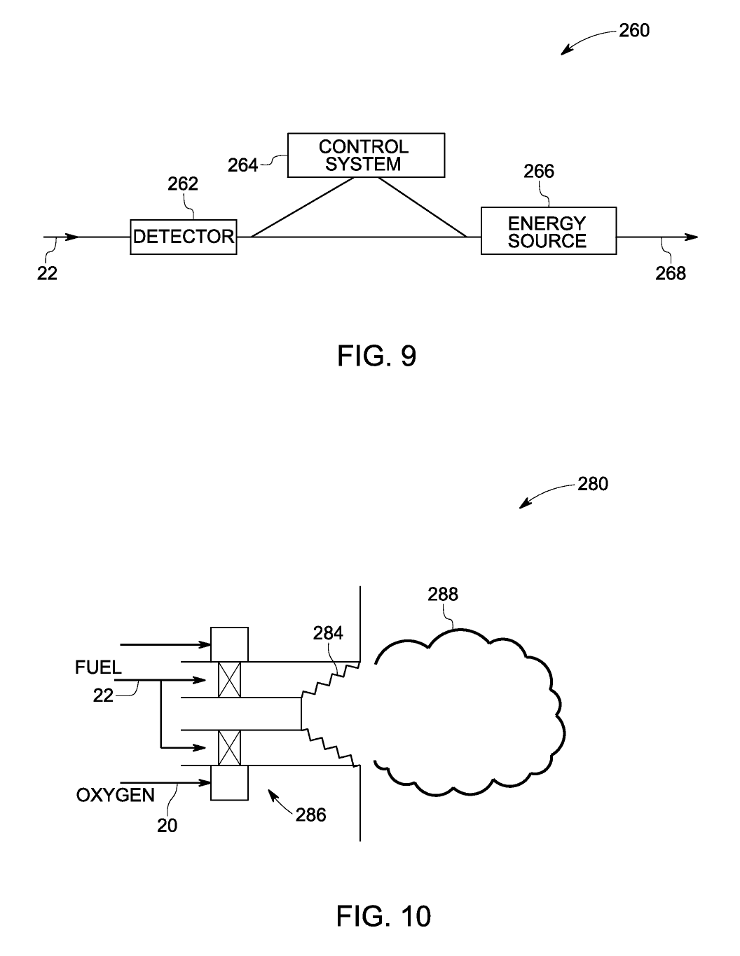

In operation, the local reforming of the fuel stream 22 in the system described above may be controlled based upon a sensed parameter. For example, the local reforming of the fuel stream 22 may be controlled based upon a fuel calorific heating value of the fuel stream 22. FIG. 9 illustrates an exemplary configuration 260 for achieving a closed-loop control of the fuel reforming based upon a sensed parameter. In the illustrated embodiment, a detector 262 is configured to measure a fuel calorific heating value of the fuel stream 22. Further, a control system 264 is coupled to the detector 262 that is operable to control the localized reforming process of the fuel stream 22 in response to the sensed fuel calorific heating value of the fuel stream 22. In particular, the control system 264 is operable to control an energy source 266 such as plasma arc, a laser, or a leaner pilot described above for controlling the localized heat addition. Again, the controlled localized heat reforming process based upon the sensed fuel calorific heating value of the fuel stream 22 facilitates generation of enhanced products 268 having a regulated composition.

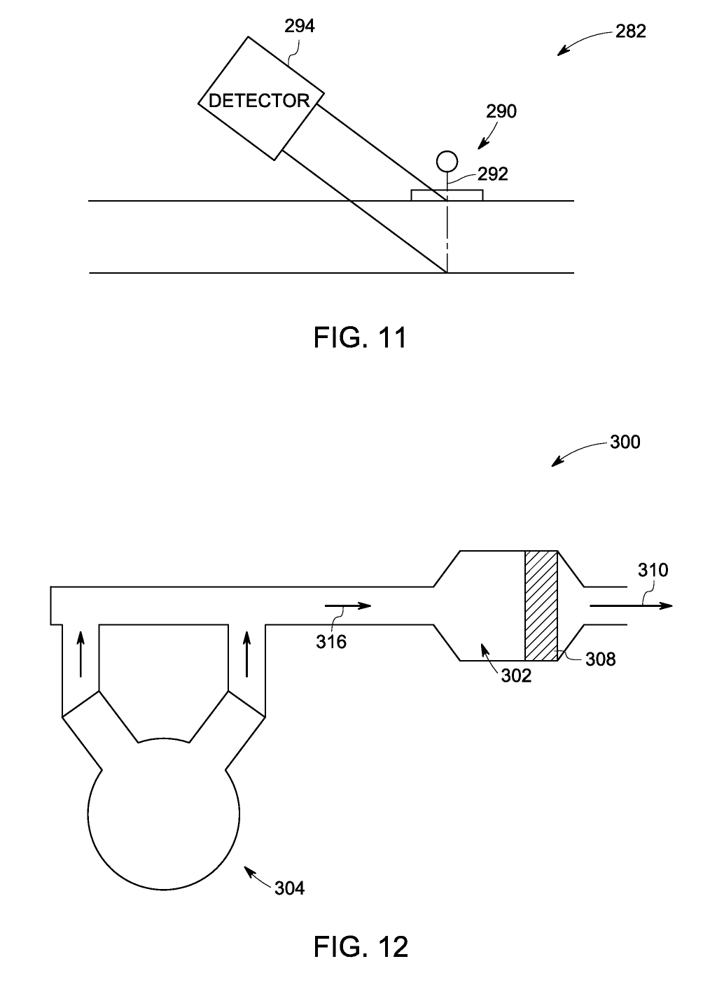

Thus, the localized heat treatment with an energy source described above may be employed for a variety of systems to achieve a local fuel reforming of the fuel stream, or for promoting formation of enhanced products such as syngas having a substantially higher carbon monoxide concentration. In certain embodiments, the energy source employed in the system may be used as an igniter, or for detecting an operational condition of the system in addition to providing the local heat addition in the heat treatment zone. FIGS. 10 and 11 illustrate exemplary configurations 280 and 282 to detect an operational condition in a system such as the syngas generator 12 of FIG. 1.

In the exemplary configuration 280 illustrated in FIG. 10, plasma arcs 284 are disposed at an exit of a premixing device 286. The premixing device 286 is configured to mix the fuel stream 22 and the oxidizer 20 to form the gaseous pre-mix 58 and may have any of the exemplary configurations such as described above with reference to FIGS. 4 and 5. In certain embodiments, the plasma arcs 284 function as igniters for igniting the gaseous pre-mix 58 and also facilitate stabilization of flame 288 in the system 280. Further, the plasma arcs 284 are configured to provide the local heat addition to the gaseous pre-mix 58 to achieve fuel reforming of the fuel stream 22. Similarly, in the exemplary configuration 282 illustrated in FIG. 11, a high-energy laser 290 is employed to achieve local fuel reforming in the system 282. Further, the laser 290 facilitates detection of an operational condition in the system. For example, the laser 290 is configured to transmit a laser sheet via a fiber optic cable 292. Further, a detector 294 may be employed to detect heavy metals and particulates within the gas stream 22. In certain embodiments, the detector 294 is configured to measure an infrared (IR) signal of hot surfaces for estimating temperature within the system.

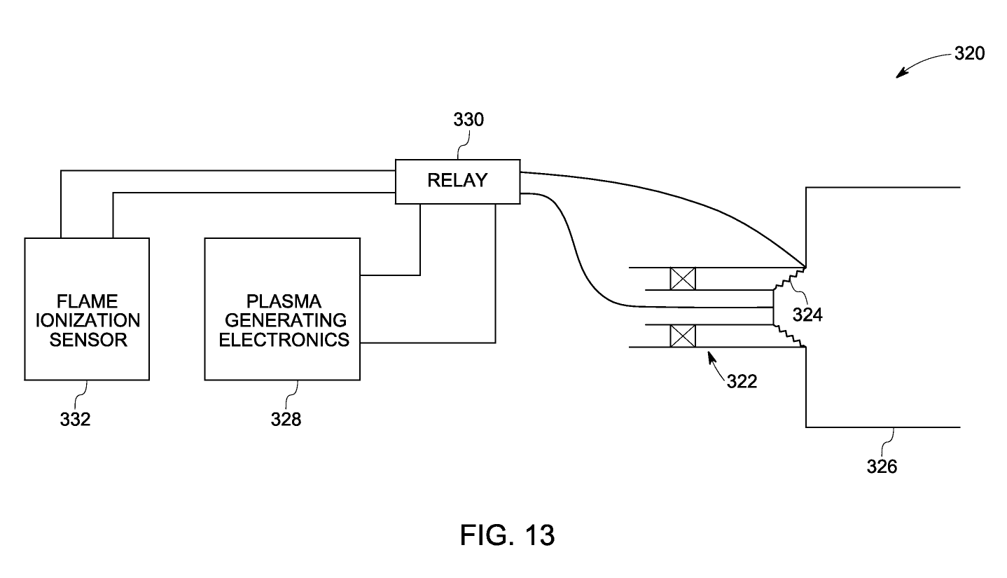

FIG. 12 illustrates an internal combustion engine system 300 having an energy source 302 coupled to an internal combustion engine 304 for achieving substantially complete combustion of exhaust products 306 from the engine 304. In the illustrated embodiment, the energy source 302 may be disposed downstream of the internal combustion engine 304 for providing local heat addition to the exhaust products 306 thereby decreasing the concentration of carbon monoxide in the exhaust products 306. Additionally, a catalytic converter 308 may be employed to further break down the exhaust products into carbon dioxide, water, and molecular nitrogen by use of catalysts. The exhaust products may be removed via an exit 310. It should be noted that the application of the energy source 302 for heat treatment of the products is required at startup to facilitate cold exhaust gas clean-up of the internal combustion engine 304, which reduces pollutant emissions during the start-up condition of the engine. Further, the energy addition system may be turned off once the catalytic converter 308 has reached normal operating temperatures.

FIG. 13 illustrates an exemplary configuration 320 for flame detection in a system such as the syngas generator 10 of FIG. 1. The system 320 includes a premixing device 322 for mixing the fuel stream 22 (see FIG. 1) and oxidizer 20. Further, an energy source such as plasma arcs 324 are disposed at an exit of the premixing device 322 for locally heating the gaseous pre-mix 58 from the premixing device 322. Subsequently, such pre-mix 58 is combusted in a combustion chamber 326. In the illustrated embodiment, the system 320 includes plasma generating electronics 328 coupled to a relay 330 and configured to generate the plasma arc 324. In addition, the system 320 includes a flame ionization sensor 332 coupled to the relay 330 for flame detection in the combustion chamber 326. Advantageously, the plasma arc 324 facilitates flame detection in the system in addition to providing the local heat treatment to the reactants. In a rich combustion system this is a system for producing enhanced products having relatively high carbon monoxide, and in a lean combustion system this provides increased flame stability and detection at ultra-lean conditions.

The various aspects of the method described hereinabove have utility in different applications such as syngas generators for enhancing the carbon monoxide concentration in an exhaust gas such as syngas. As noted above, the localized heat treatment of the premixed reactants in syngas generators shifts the equilibrium point away from carbon dioxide formation thereby promoting formation of an increased amount of carbon monoxide (CO) in the syngas. Furthermore, the technique described hereinabove may be employed to achieve staged heat treatment of an exhaust gas from a system to generate enhanced products. In addition, the energy sources described above such as laser, plasma and so forth also facilitate dynamics stabilization within the system and diagnostics and control of such systems as described above. Advantageously, the localized heat treatment may be employed for a vast range of applications for enhancing carbon monoxide concentrations in a rich exhaust gas or by facilitating more complete combustion of the fuel to CO2 and H2O in a lean exhaust gas, and providing increased flame stability in either systems.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A device for enhancing carbon monoxide (CO) production in an exhaust gas generated from a system, comprising:
a flow path configured to direct the exhaust gas from the system into a heat treatment zone; and
an energy source configured to provide local heat treatment to the exhaust gas in the heat treatment zone for shifting an equilibrium point of reaction substantially away from carbon dioxide (CO2) formation thereby promoting formation of an increased level of carbon monoxide (CO) in the exhaust gas.

2. The device of claim 1, wherein the system comprises a syngas generator of a gas to liquid system, or a gasifier and the exhaust gas comprises syngas.

3. The device of claim 1, wherein the energy source is configured to shift the equilibrium point of premixed reactants in the heat treatment zone located at an exit of a premixing zone of the system.

4. The device of claim 1, wherein the energy source is configured to shift the equilibrium point of the exhaust gas in the heat treatment zone located at an exit of a combustion zone of the system.

5. The device of claim 1, wherein the energy source comprises one or more plasma arcs, or a laser, or a leaner pilot, or combinations thereof.

6. The device of claim 1, wherein the device is configured to facilitate exhaust stream cleanup of an engine using excess air in the stream to enhance CO₂ and water formation and to remove pollutant emissions during a start-up condition of the engine.

7. The device of claim 1, wherein the device is configured to detect an operational condition of the system.

8. The device of claim 7, wherein the operational condition comprises a flame detection condition, or a flashback condition in the system.

9. The device of claim 7, wherein the operational condition comprises detection of a proportion of solids within the fuel stream or exhaust of a gasifier.

10. A method of producing synthesis gas (syngas), comprising:
premixing a fuel stream and oxidizer to form a gaseous premix in a premixing zone;
locally heating the gaseous premix downstream of the premixing zone; and
combusting the gaseous premix in a combustion zone to form syngas enriched with carbon monoxide (CO).

11. The method of claim 10, wherein locally heating the gaseous pre-mix comprises heating the gaseous pre-mix at the exit of the premixing zone via an energy source, or via a trapped vortex configuration, or combinations thereof.

12. The method of claim 11, wherein the energy source comprises one or more plasma arcs, or a laser, or a leaner pilot, or combinations thereof.

13. The method of claim 10, comprising shifting an equilibrium point of reaction in the combustion zone substantially away from carbon dioxide (CO₂) formation to enable formation of syngas enriched with carbon monoxide (CO).

14. The method of claim 10, further comprising heating the syngas at an exit of the combustion zone.

15. A system for producing syngas, comprising:
a premixing device configured to mix a fuel stream and an oxidizer in a premixing zone to form a gaseous pre-mix;
an energy source coupled to the premixing device and configured to locally shift the equilibrium point of the gaseous pre-mix in a heat treatment zone disposed downstream of the premixing zone; and
a combustion zone configured to receive the gaseous pre-mix from the heat treatment zone and to combust the gaseous premix to produce syngas enriched with carbon monoxide (CO).

16. The system of claim 15, wherein the system comprises a syngas generator in a gas-to-liquid system.

17. The system of claim 15, wherein the fuel stream comprises a hydrocarbon fuel, or steam, or a tail gas, or combinations thereof and the oxidizer comprises oxygen.

18. The system of claim 15, wherein the energy source comprises one or more plasma arcs, or a laser, or a leaner pilot, or combinations thereof.

19. The system of claim 18, wherein the laser is configured to facilitate flame detection in the system by sensing flame radiation within the system via a fiber optic cable.

20. The system of claim 18, wherein the laser is configured to facilitate system life monitoring by sensing surface temperatures within the system via a fiber optic cable.

21. The system of claim 18, wherein the plasma arcs are configured to facilitate dynamics stabilization in the system by flame ionization sensing and plasma arc stabilization.

22. The system of claim 18, wherein the laser is configured to detect a proportion of solids within the fuel or exhaust stream in a gasifier.

23. The system of claim 15, further comprising a control system configured to control operation of the energy source based upon a sensed parameter.

24. The system of claim 22, wherein the sensed parameter comprises a fuel calorific heating value of the fuel stream.
